(19) United States
(12) Patent Application Publication
(10) Pub. No.: US 2024/0108733 A1
(43) Pub. Date: Apr. 4, 2024

(54) PRODRUGS OF PHARMACEUTICAL AGENTS

(71) Applicants: ORN ALMARSSON, CAMBRIDGE, MA (US); CARLOS N. SANRAME, LEXINGTON, MA (US)

(72) Inventors: ORN ALMARSSON, CAMBRIDGE, MA (US); CARLOS N. SANRAME, LEXINGTON, MA (US)

(21) Appl. No.: 18/269,640

(22) PCT Filed: Dec. 24, 2021
(86) PCT No.: PCT/US2021/065175

§ 371 Date: Jun. 26, 2023

Related U.S. Application Data
(60) Provisional application No. 63/130,435, filed on Dec. 24, 2020.

(30) Foreign Application Priority Data
Dec. 22, 2021 (WO) PCT/US2021/064918

(51) Int. Cl. A61K 47/54

(52) U.S. Cl. CPC A61K 47/545

(57) ABSTRACT

The present invention provides for novel prodrugs and compositions comprising the prodrugs as well methods of making and using the same. The compositions include pharmaceutical compositions comprising a prodrug.

### PRIORITY

This application claims priority to U.S. Provisional Application No. 63/130,435, filed on 24 Dec. 2020, and PCT Application PCT/US21/64918, filed on 22 Dec. 2021, each of which are hereby incorporated by reference in their entireties.

### FIELD OF THE INVENTION

The field of the invention relates to derivatives, including prodrugs, of active pharmaceutical ingredients.

### BACKGROUND OF THE INVENTION

Prodrugs have an important role in medicine and health. Many drugs are optimally delivered to the body in the form of a prodrug, which is activated following delivery to unmask the drug. Design of prodrugs has been carried out over decades (see for example reviews by Stella 2020
https://pubmed.ncbi.nlm.nih.gov/33002466/, Rautio 2008
https://pubmed.ncbi.nlm.nih.gov/18219308/, Rautio 2018
https://pubmed.ncbi.nlm.nih.gov/29700501/, and Huttunen 2011
https://pubmed.ncbi.nlm.nih.gov/21737530/; each of which are hereby incorporated by reference in their entireties.)

Through the years, many pro-moieties (i.e. chemical functionalities added to a drug to make prodrugs) have been explored. A particular problem existing in the art is that prodrugs that have basic groups likely have pharmacology, as heterocyclic basic drugs are common and few amine bases are usable in comparison with non-basic and acidic moieties, such as alcohols (e.g. ethanol in ACE inhibitor enalapril) or carboxylic acids (such as acetic acid in acetyl salicylic acid). While salt forming agents like ethanolamines are occasionally used in pharmaceutical salts, these bases are generally not part of the prodrug repertoire. As an example of basic pro-moiety, nicotinic acid is a heterocyclic base which is a drug for cardiovascular conditions, which has been proposed as a prodrug function despite being a vasodilator and having potential to negatively impact liver function.

### SUMMARY OF THE INVENTION

The present invention is directed towards new forms of an active pharmaceutical ingredient (“API”) that has improved properties including improved physicochemical characteristics as well as methods for the preparation of compounds. In one aspect, the invention provides for a prodrug compound, wherein the compound comprises an API with a hydrogen substituted for a prodrug moiety selected from any one of R10-R105. The invention further provides for salts, co-crystals, geometric isomers and stereoisomers of the prodrug compound. The invention further provides for pharmaceutical compositions comprising a prodrug of the present invention and a pharmaceutically acceptable excipient. Another aspect provides for a method of treating a subject with a prodrug of the present invention, wherein the subject has a disease or disorder for which the parent API is indicated, said method comprising the step of administering to said subject an effective amount of said prodrug.

### DETAILED DESCRIPTION OF THE INVENTION

### Definitions

The term “active pharmaceutical ingredient” or “API” refers to the chemical substance that exerts the desired pharmacological action of a drug product, i.e., the free form (neutral, free acid or free base) of an active ingredient of a drug product as administered.

The term “comprising”, as used herein, is synonymous with “including,” “containing,” or “characterized by,” is inclusive or open-ended and does not exclude additional, unrecited elements or method steps, unless the context clearly requires otherwise. For example, a composition of the present invention “comprising an active ingredient” contains one or any number of active ingredients, unless otherwise specified.

The phrases “consists of” or “consisting of” are closed-ended and includes only those features specified. When used in a clause, the phrases “consists of” or “consisting of” limit only the element set forth in that clause.

The phrases “consists essentially of” and “consisting essentially of are partially open and limited to features that do not materially affect the basic and novel characteristic(s)” of the claimed invention. For example, the phrases include an unrecited level of impurities that do not materially affect the basic and novel characteristic(s), e.g., activity or stability, of a composition of the invention.

As used herein, when a range is set forth as “between” two values, it is understood that the range is inclusive of the end values.

As used herein, the terms “treat”, “treating” or “treatment” means to alleviate, reduce or abrogate one or more symptoms or characteristics of a disease or condition and may be curative, palliative, prophylactic or slow the progression of the disease. The term “therapeutically effective amount” is intended to mean that amount of drug that will elicit a desired biological or pharmacological response, i.e., an amount sufficient to treat said disease.

The term “effective amount” means an amount of active ingredient(s) that will result in a desired effect or result. The term “therapeutically effective amount” means an amount of active ingredient(s) that will elicit a desired biological or pharmacological response, e.g., effective to prevent, alleviate, or ameliorate symptoms of a disease or disorder; slow, halt or reverse an underlying process or progression of a disease or disorder; partially or fully restore cellular function; or prolong the survival of the subject being treated.

The term “subject” means an animal, including mammals, non-human animals, and especially humans. In one embodiment, the subject is a human. In another embodiment, the subject is a human male and in another, the subject is a human female. Further, a “subject” can include a patient afflicted with or suspected of being afflicted with a condition or disease. Thus, the terms “subject” and “patient” may be used interchangeably herein.

The term “excipient” refers to a pharmaceutically acceptable, inactive substance used as a carrier for the pharmaceutically active ingredient(s) and includes antiadherents, binders, coatings, disintegrants, fillers, diluents, flavors, bulkants, colours, glidants, dispersing agents, wetting agents, lubricants, preservatives, sorbents and sweeteners.

The term “unit dose” refers to the amount of prodrug administered to a subject in a single dose.

The term “prodrug” as used herein refers to an API that is covalently modified with a prodrug moiety. After administration, the prodrug is chemically or enzymatically metabolized in vivo resulting in the formation of the parent API. The prodrugs of the present invention may, in one embodiment, be biologically active or, in another embodiment, be biologically inactive in their prodrug form; wherein the biological activity is the biological activity of the parent drug. In either case, the prodrug is metabolized in vivo into the parent drug.

The term “prodrug moiety” is the non-API portion of a prodrug, i.e., a molecule that is appended to an API, typically through the substitution of a hydrogen atom on the API to form an ester or other covalent bond that is metabolized in vivo into the API (parent drug).

### Compositions

In one aspect, the invention provides for an API substituted with a prodrug moiety, wherein said prodrug moiety is selected from any one of R10-R105.

In a preferred embodiment, the API is substituted with a prodrug moiety selected from R10-R22.

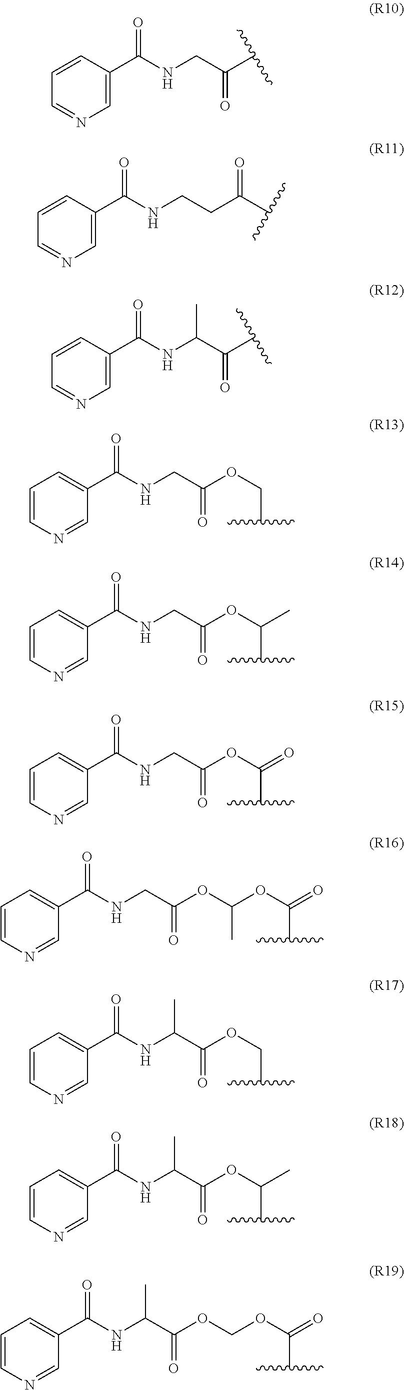

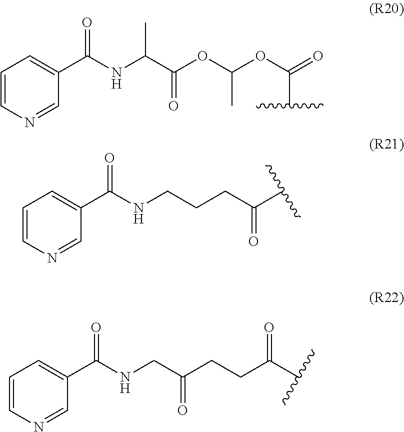 [SMILES: *C(=O)OC(C)OC(=O)C(C)NC(=O)c1cccnc1 ; *C(=O)CCCNC(=O)c1cccnc1 ; *C(=O)CCC(=O)CNC(=O)c1cccnc1]

In another embodiment, the API is substituted with a prodrug moiety selected from any one of R23-R37.

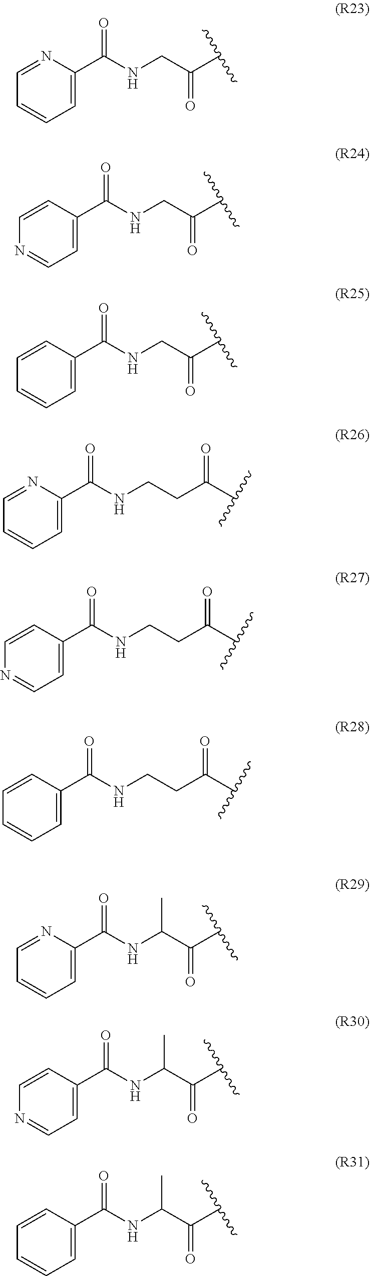

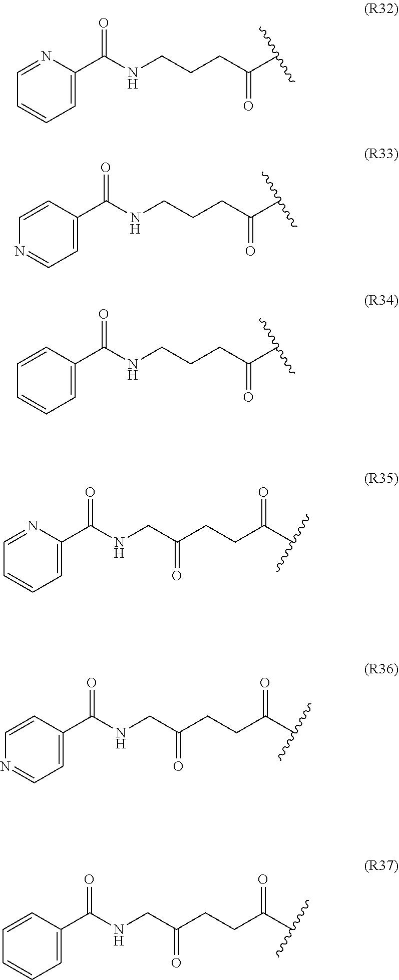 [SMILES: *C(=O)CCCNC(=O)c1ccccn1 ; *C(=O)CCCNC(=O)c1ccncc1 ; *C(=O)CCCNC(=O)c1ccccc1 ; *C(=O)CCC(=O)CNC(=O)c1ccccn1 ; *C(=O)CCC(=O)CNC(=O)c1ccncc1 ; *C(=O)CCC(=O)CNC(=O)c1ccccc1]

In a preferred embodiment, the API is substituted with a prodrug moiety selected from any one of R38-R54.

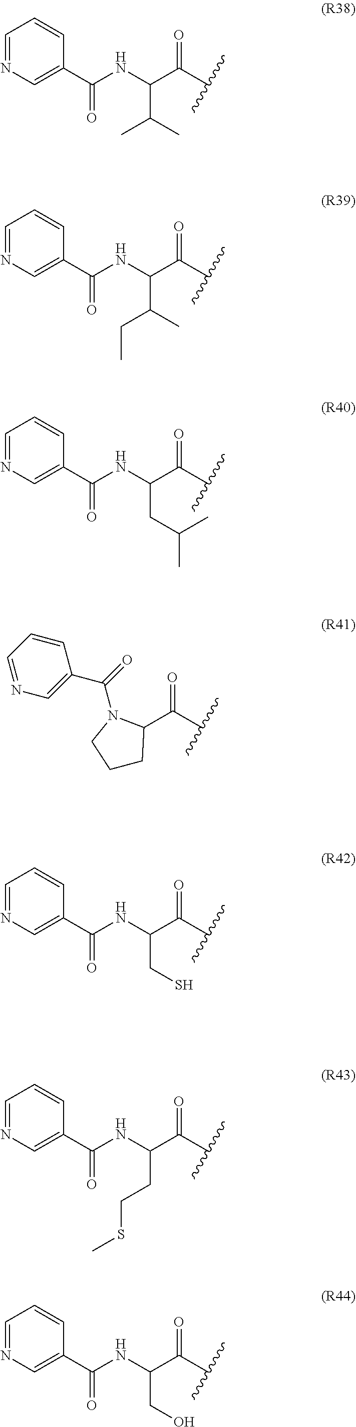

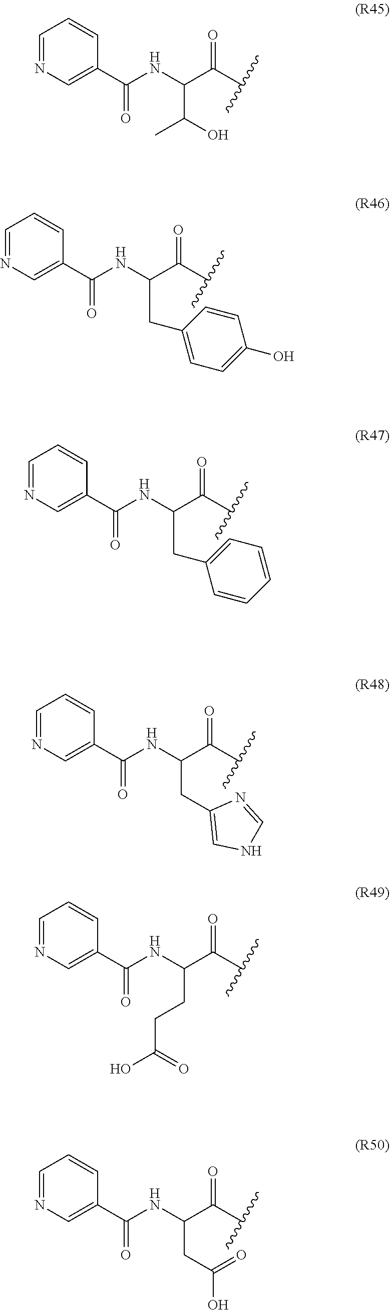 [SMILES: *C(=O)C(NC(=O)c1cccnc1)C(C)O ; *C(=O)C(Cc1ccc(O)cc1)NC(=O)c1cccnc1 ; *C(=O)C(Cc1ccccc1)NC(=O)c1cccnc1 ; *C(=O)C(Cc1c[nH]cn1)NC(=O)c1cccnc1 ; *C(=O)C(CCC(=O)O)NC(=O)c1cccnc1 ; *C(=O)C(CC(=O)O)NC(=O)c1cccnc1]

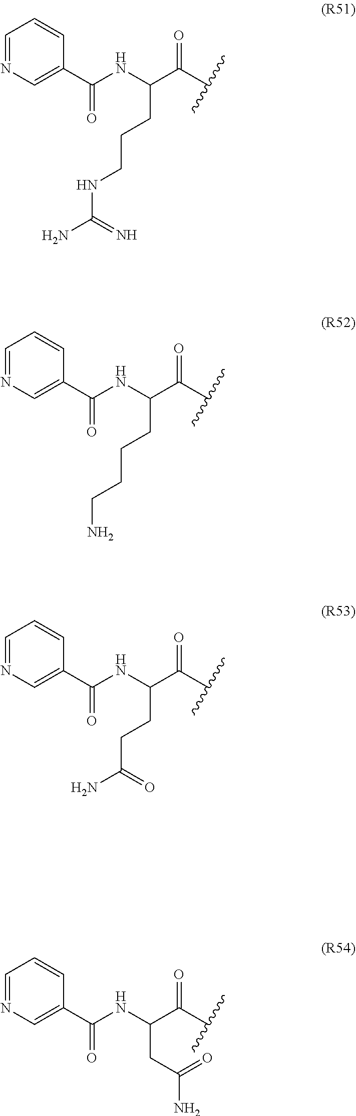 [SMILES: *C(=O)C(CCCNC(=N)N)NC(=O)c1cccnc1 ; *C(=O)C(CCCCN)NC(=O)c1cccnc1 ; *C(=O)C(CCC(N)=O)NC(=O)c1cccnc1 ; *C(=O)C(CC(N)=O)NC(=O)c1cccnc1]

In another embodiment, the API is substituted with a prodrug moiety selected from any one of R55-R71.

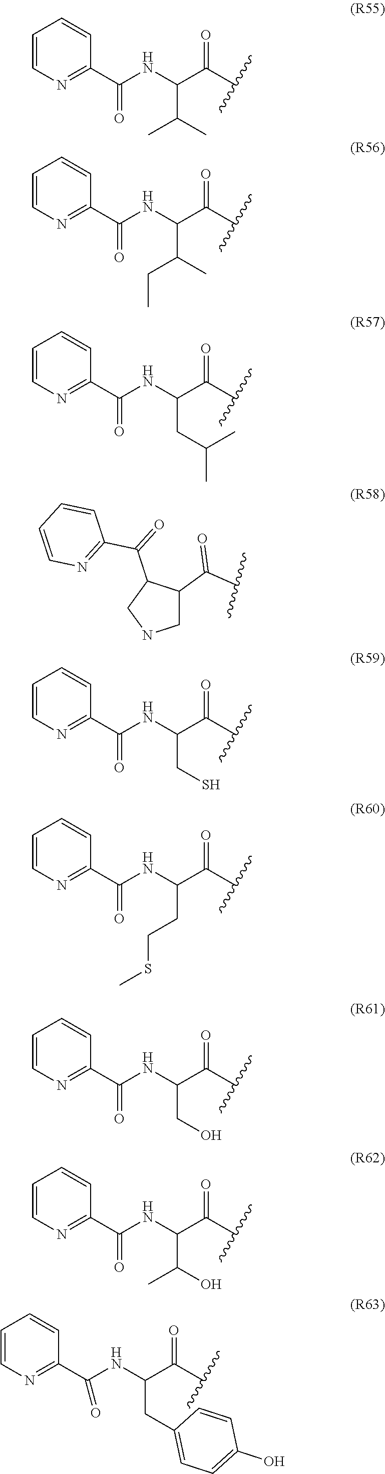

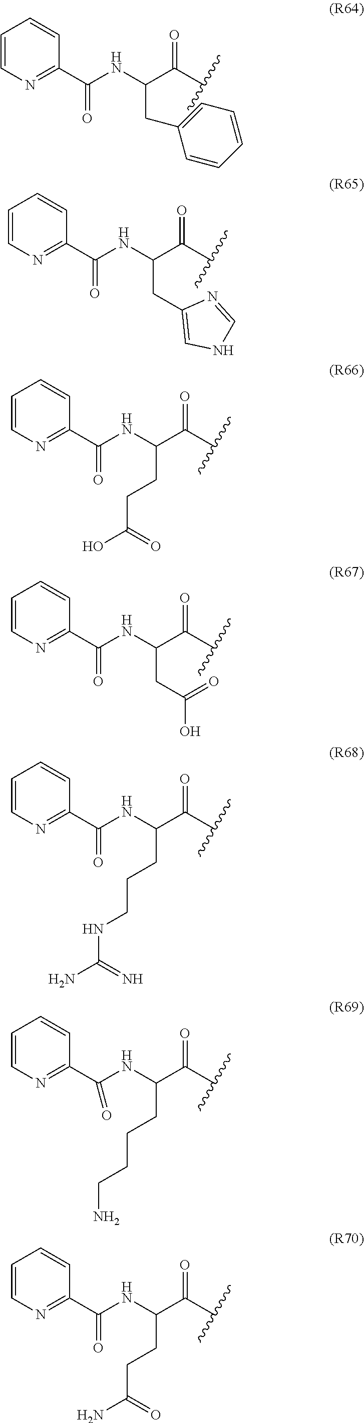

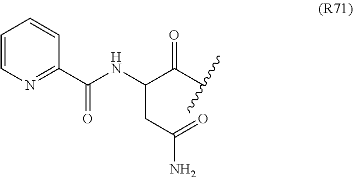 [SMILES: *C(=O)C(CC(N)=O)NC(=O)c1ccccn1]

In another embodiment, the API is substituted with a prodrug moiety selected from any one of R72-R88.

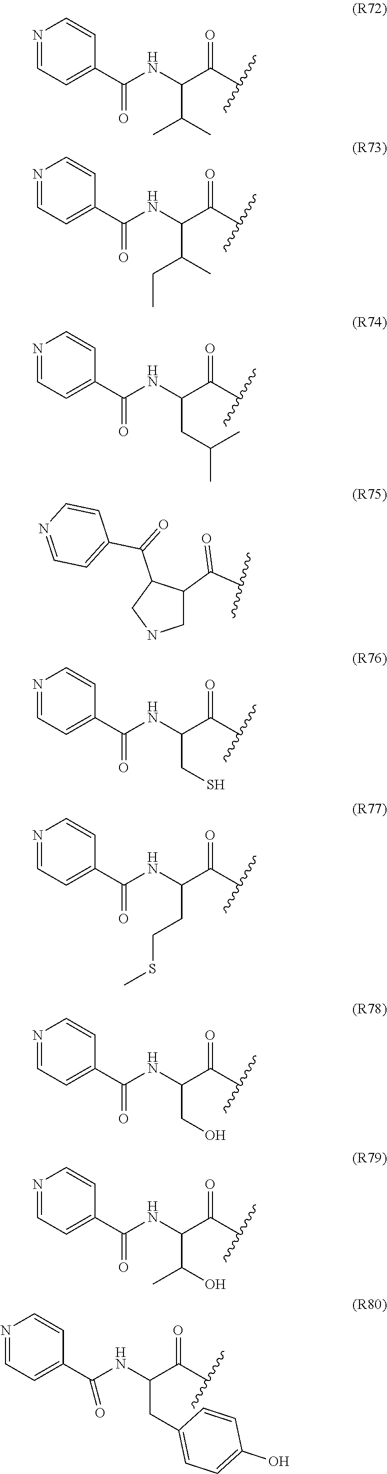

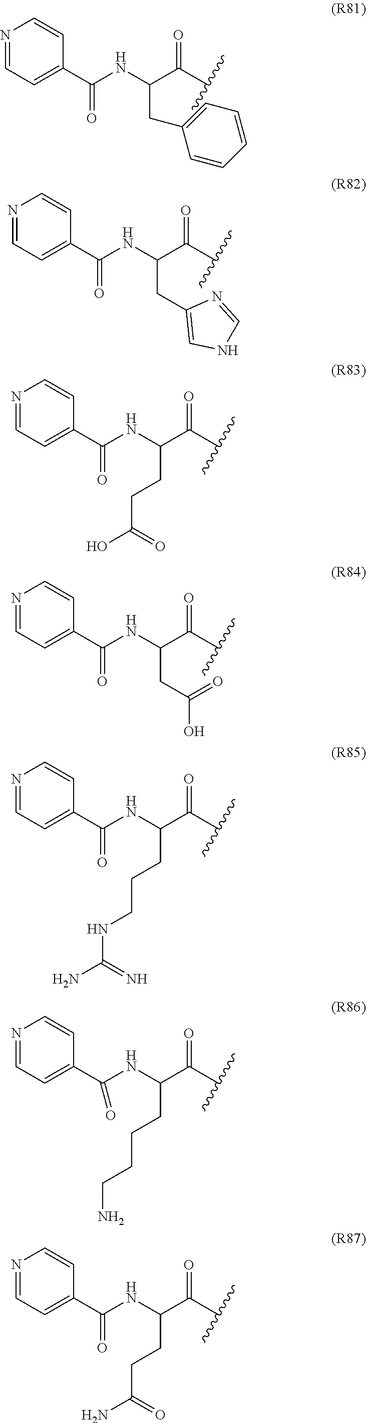

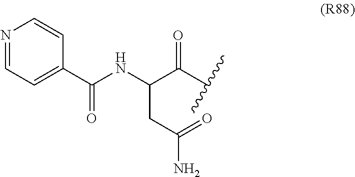 [SMILES: *C(=O)C(CC(N)=O)NC(=O)c1ccncc1]

In another embodiment, the API is substituted with a prodrug moiety selected from any one of R89-105.

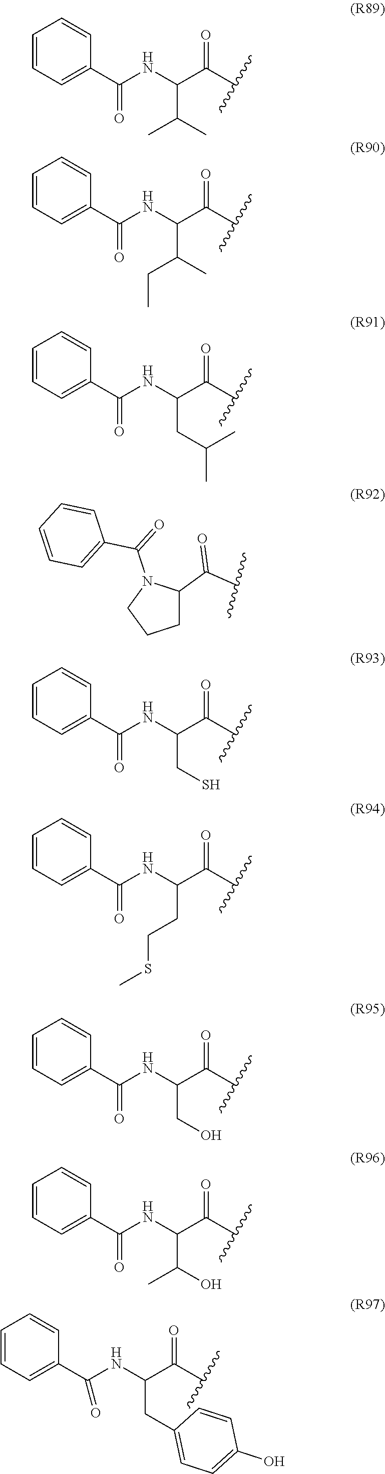

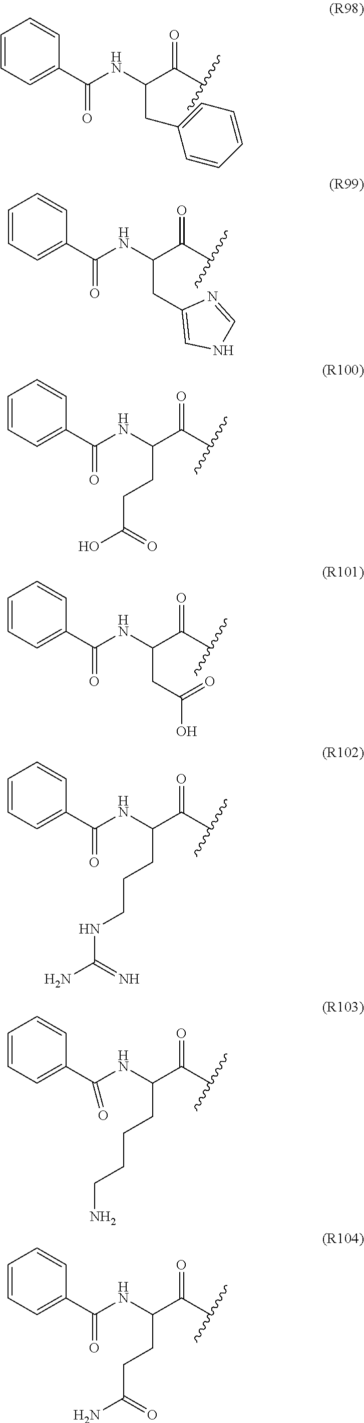

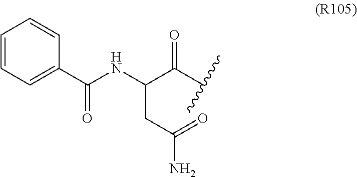 [SMILES: *C(=O)C(CC(N)=O)NC(=O)c1ccccc1]

In a more preferred embodiment, the prodrug of the present invention is a nicotinuric acid prodrug of an API where the prodrug moiety is R10. Nicotinuric acid (I), also referred to as nicotinoyl glycine, is a metabolite of nicotinic acid, has limited pharmacology and is renally excreted.

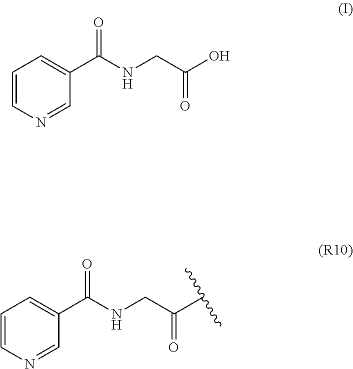 [SMILES: O=C(O)CNC(=O)c1cccnc1 ; *C(=O)CNC(=O)c1cccnc1]

To date, there is no mention of the use of this moiety (nicotinuric acid) in prodrug literature, to the knowledge of the inventors. The reduced pharmacology and liver concern with this compound are advantageous to the use in prodrug with nucleophilic groups like amines, thiols and alcohols, for example. Advantages can be in various routes of administration, dosing paradigms and formulations. For example, oral delivery of a low-solubility drug with an —OH or similar function can be facilitated by appending, i.e., covalently bonding, a nicotinuric acid substituent to make, e.g., an ester, producing a weak base prodrug molecule that can be protonated in the stomach for facilitated dissolution and better bioavailability from the gut, relative to the parent drug.

In prodrugs where a hydrogen on an —OH of an API is substituted with R10, the acetyl extension of the nicotinic amide may offer an advantage for cleavage at the ester bond indicated with a wavy line in compounds (II) and (III); this ester bond is less encumbered than the ester of nicotinic acid, which can improve the bioactivation required to liberate the drug in the body.

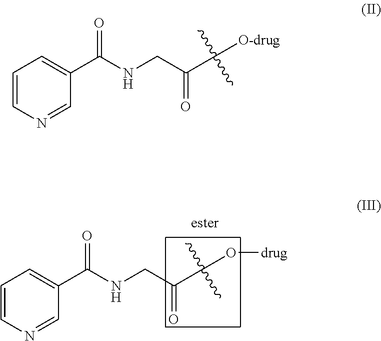

In one embodiment, a hydrogen on the API is substituted with a prodrug moiety selected from any one of R10-R105. In a further embodiment, the hydrogen is a hydrogen of a functional group selected from the group consisting of: hydroxy, sulfonamide, sulfamate, imide, amidine, guanidine, urea, hydroxy-urea, acylsulfonamide, thio, carbamate, primary amine, secondary amine, primary amide, secondary amide, imine, hemi-aminal, hydroxylamine or hydroxylamide. In one embodiment, the functional group is selected from the group consisting of: hydroxy, sulfonamide, sulfamate, imide, amidine, guanidine, urea, hydroxy-urea, thiol and acylsulfonamide. In another embodiment, the functional group is selected from the group consisting of: carbamate, primary amine, secondary amine, primary amide, secondary amide, imine, hemi-aminal, hydroxylamine or hydroxylamide. In one embodiment, the substituted functional group is —OH. In another embodiment, the substituted functional group is —S(O)₂NH. In another embodiment, the substituted functional group is —NH₂. In another embodiment, the substituted functional group is a secondary amine. In another embodiment, the substituted functional group is —C(O)NH₂. In another embodiment, the substituted functional group is a secondary amide. In another embodiment, the substituted functional group is ═NH. In another embodiment, the substituted functional group is ═NOH. In another embodiment, the substituted functional group is ═NOH. In another embodiment, the substituted functional group is —S(═O)₂NHC(═O). In another embodiment, the substituted functional group is —S(═O)₂NHC(═O). In another embodiment, the substituted functional group is —C(═O)NOH.

In one aspect, the prodrug has a formula R1-Z—R2, wherein: R1 is a prodrug moiety selected from any one of R10-R105 and together —Z—R2 is an API radical, wherein —Z— is —O— or a di-radical of a sulfur or nitrogen atom in a functional group. In one embodiment, —Z— is a di-radical of a functional group selected from hydroxy, sulfonamide, sulfamate, imide, amidine, guanidine, urea, hydroxy-urea, acylsulfonamide, thio, carbamate, primary amine, secondary amine, primary amide, secondary amide, imine, hemi-aminal, hydroxylamine or hydroxylamide.

In one aspect, the prodrug has a formula R1-R2, wherein: R1 is a prodrug moiety selected from any one of R10-R105; and —R2 is an API comprising a radical. In one embodiment the radical is on a functional group selected from the group consisting of: hydroxy, sulfonamide, sulfamate, imide, amidine, guanidine, urea, hydroxy-urea, acylsulfonamide, thio, carbamate, primary amine, secondary amine, primary amide, secondary amide, imine, hemi-aminal, hydroxylamine and hydroxylamide. For prodrugs of the formula R1-R2, R1 is a substitution for a hydrogen. In one embodiment, the substitution is for a hydrogen on a functional group selected from the group consisting of: hydroxy, sulfonamide, sulfamate, imide, amidine, guanidine, urea, hydroxy-urea, acylsulfonamide, thiol, carbamate, primary amine, secondary amine, primary amide, secondary amide, imine, hemi-aminal, hydroxylamine and hydroxylamide.

In one aspect, the prodrug has a formula (R1)₂-R2, wherein: R1 is a prodrug moiety independently selected from any one of R10-R105; and —R2 is an API comprising a di-radical. In one embodiment the di-radical is on two functional groups independently selected from the group consisting of: hydroxy, sulfonamide, sulfamate, imide, amidine, guanidine, urea, hydroxy-urea, acylsulfonamide, thio, carbamate, primary amine, secondary amine, primary amide, secondary amide, imine, hemi-aminal, hydroxylamine and hydroxylamide. For prodrugs of the formula (R1)₂-R2, each R1 is a substitution for a hydrogen. In one embodiment, each substitution is for a hydrogen on a functional group independently selected from the group consisting of: hydroxy, sulfonamide, sulfamate, imide, amidine, guanidine, urea, hydroxy-urea, acylsulfonamide, thiol, carbamate, primary amine, secondary amine, primary amide, secondary amide, imine, hemi-aminal, hydroxylamine and hydroxylamide. In one embodiment, each R1 is identical, i.e., the same prodrug moiety. In another embodiment, each functional group substituted with a prodrug moiety is the same.

In one aspect, the prodrug has a formula (R1)₃-R2, wherein: each R1 is a prodrug moiety independently selected from any one of R10-R105; and —R2 is an API comprising a tri-radical. In one embodiment each radical is on a functional group independently selected from the group consisting of: hydroxy, sulfonamide, sulfamate, imide, amidine, guanidine, urea, hydroxy-urea, acylsulfonamide, thio, carbamate, primary amine, secondary amine, primary amide, secondary amide, imine, hemi-aminal, hydroxylamine and hydroxylamide. For prodrugs of the formula (R1)₃-R2, each R1 is a substitution for a hydrogen. In one embodiment, each substitution is for a hydrogen on a functional group independently selected from the group consisting of: hydroxy, sulfonamide, sulfamate, imide, amidine, guanidine, urea, hydroxy-urea, acylsulfonamide, thiol, carbamate, primary amine, secondary amine, primary amide, secondary amide, imine, hemi-aminal, hydroxylamine and hydroxylamide. In one embodiment, each R1 is identical, i.e., the same prodrug moiety. In another embodiment, each functional group substituted with a prodrug moiety is the same.

In one aspect, the invention provides for a prodrug of a API (parent drug) selected from the group consisting of: dolutegravir, bictegravir, acyclovir, tenofovir, docosanol, baloxavir, famciclovir, vidarabine, cytarabine, amprenavir, saquinavir, entecavir, penciclovir, clevudine, trifluridine, laninamivir, edoxudine, zanamivir, amsacrine, ribavirin, idoxuridine, gancicolvir, ledipasvir, peramivir, imiquimod, adefovir, floxuridine, 5-fluorouridine, cidofovir, oseltamivir, zidovudine, chlormetacrine, nelfinavir, tiazofurin, rilpivirine, abacavir, dideoxyadenosine, indinavir, tipranavir, didanosine, simeprevir, dasabuvir, thymine riboside, glecaprevir, ombitasvir, paritaprevir, grazoprevir, darunavir, beclabuvir, emtricitabine, sofosbuvir, voxilaprevir, voriconazole, fluconazole, isovuconazole, ravuconazole, buclosamide, clofenesin, cloxyquin, viridine, ciclopriox, siccanin, efinaconazole, metconazole, sphingofungin, cyproconazole, parabens, sinefungin, albofungin, mycosamine, chlorquinaldol, propofol, tapentadol, armepavine, topiramate, amodiaquin, droloxifene (hydroxy tamoxifene), venlafaxine, resveratrol, morphine, docosanol, bictegravir, linezolid, aripiprazole hydroxymethyl, dehydro-aripiprazole hydroxymethyl, brexpiprazole hydroxymethyl, lenalinomide, suberoylanilide hydroxamic acid, zileuton, cimetidine, famotidine, norgestimate, samidorphan, imolamine, tipiracil, ceftazidime, felbamate, selexipag, cannabidiol, deoxycholic acid, apomorphine, albuterol, cortisone, corticosterone, prednisone, formoterol, and salmeterol; wherein a hydrogen on said API is substituted with a prodrug moiety selected from R10-R105. In one embodiment, the prodrug moiety is a substitution of a hydrogen bonded to an oxygen, nitrogen or sulfur atom. In a further embodiment, the prodrug moiety substitutes a hydrogen on a functional group selected from any one of: hydroxy, sulfonamide, sulfamate, imide, amidine, guanidine, urea, hydroxy-urea, acylsulfonamide, thio, carbamate, primary amine, secondary amine, primary amide, secondary amide, imine, hemi-aminal, hydroxylamine or hydroxylamide. In a preferred embodiment, the functional group is selected from any one of: hydroxy, sulfonamide, sulfamate, imide, amidine, guanidine, urea, hydroxy-urea, acylsulfonamide and thiol.

In one embodiment, the prodrug moiety is selected from any one of R23-R37. In another embodiment, the prodrug moiety is selected from any one of R55-R71. In another embodiment, the prodrug moiety is selected from any one of R72-R88. In another embodiment, the prodrug moiety is selected from any one of R89-105. In a preferred embodiment, the prodrug moiety is selected from R10-R22. In a preferred embodiment, the prodrug moiety is selected from any one of R38-R54. In a more preferred embodiment, the prodrug moiety is R10. In one embodiment, the API is substituted with one prodrug moiety. In another embodiment, the API is substituted with two prodrug moieties. In a third embodiment, the API is substituted with three. Preferably, where a API is substituted with two or three prodrug moieties, the prodrug moieties are the same.

In one embodiment, the prodrug has a formula R1-Z—R2, wherein —R2 is selected from any one of R200-R206, R211-R213, R216-R221, R223, wherein the dashed line ( - - - or [symbol] ) represents a bond, wherein R1- is a prodrug moiety selected from any one of R10-R105, and wherein —Z— is —O—.

In one embodiment, the prodrug has a formula (R1-Z)₂—R2, wherein —R2 is selected from any one of R208, R209, R210, R214, R222 or R224, wherein the dashed line ( [symbol] ) represents a bond, wherein R1- is a prodrug moiety selected from any one of R10-R105, and wherein —Z— is —O—.

In one embodiment, the prodrug has a formula (R1-Z)₃—R2, wherein —R2 is R207 or R215, wherein the dashed line ( [symbol] ) represents a bond, wherein, alternatively, R1-Z— together is —OH, or R1- is a prodrug moiety selected from any one of R10-R105, and —Z— is —O—, with the proviso that not more than two R1-Z— together are —OH. In a preferred embodiment, for at least two R1-Z— groups, and more preferred all three R1-Z— groups, R1- is a prodrug moiety selected from any one of R10-R105 and —Z— is —O—.

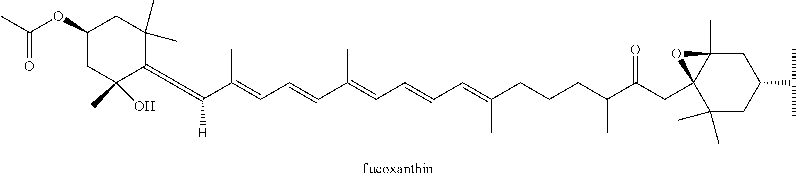

In one embodiment, the prodrug has a formula R1-Z—R2, wherein —R2 is R225 or R226, wherein the dashed line ( [symbol] ) represents a bond, wherein R1- is a prodrug moiety selected from any one of R10-R105, and wherein —Z— and is —NH—.

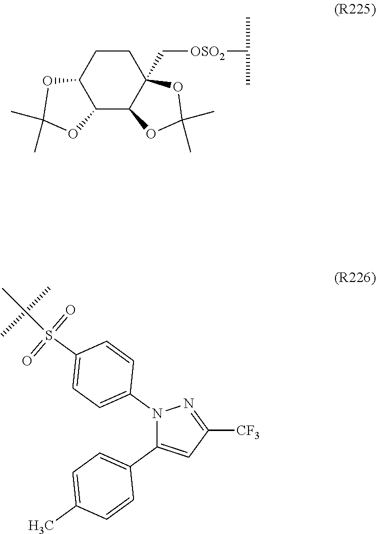

In one embodiment, the prodrug has a formula R1-Z—R2, —R2 is R227, wherein the dashed line ( [symbol] ) represents a bond, wherein R1- is a prodrug moiety selected from any one of R10-R105, and wherein —Z— and is —S—.

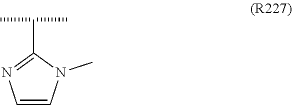

In one embodiment, the prodrug has a formula R1-Z—R2, wherein —R2 is R228, wherein the dashed line ( [symbol] ) represents a bond, wherein together R1-Z— is —N(R1)C(═O)NMe₂, and wherein R1 is a prodrug moiety selected from any one of R10-R105.

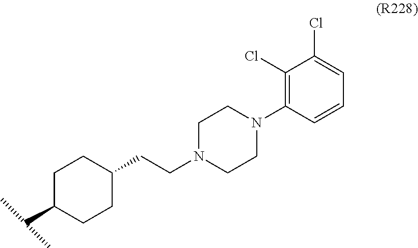

In embodiment, the prodrug has a formula R1-R2, wherein R1- is a prodrug moiety selected from any one of R10-R105 and R2 is any one of: R230-R251, wherein the dashed line ( [symbol] ) is a bond.

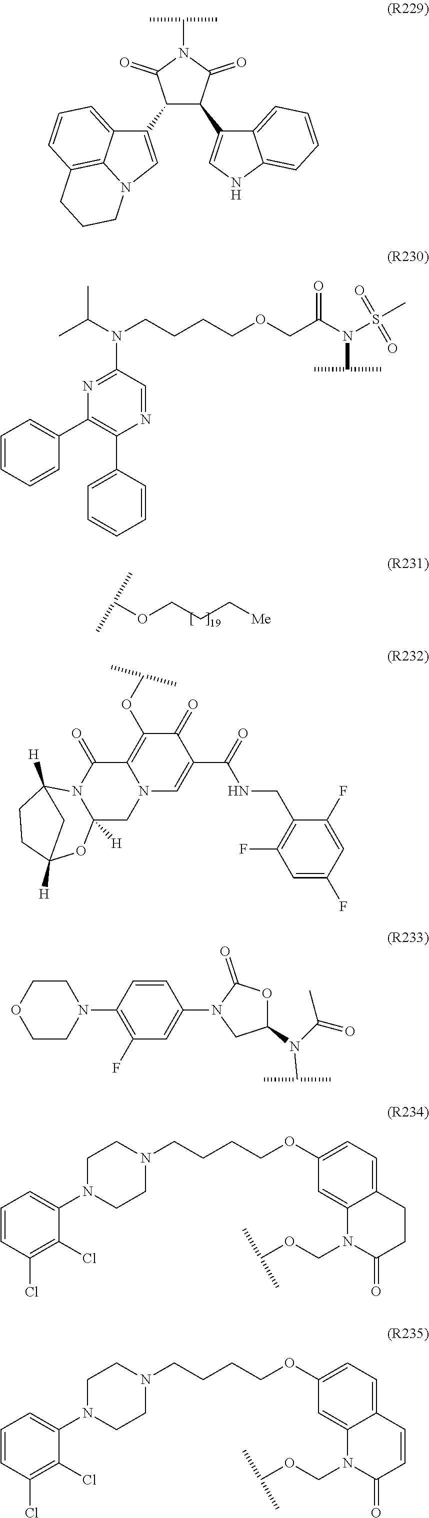

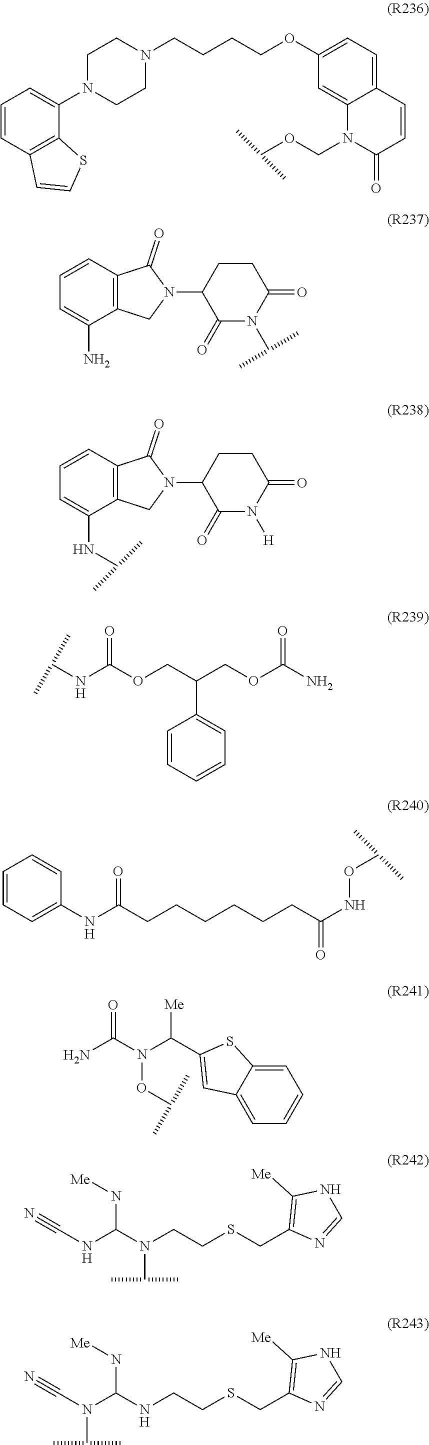

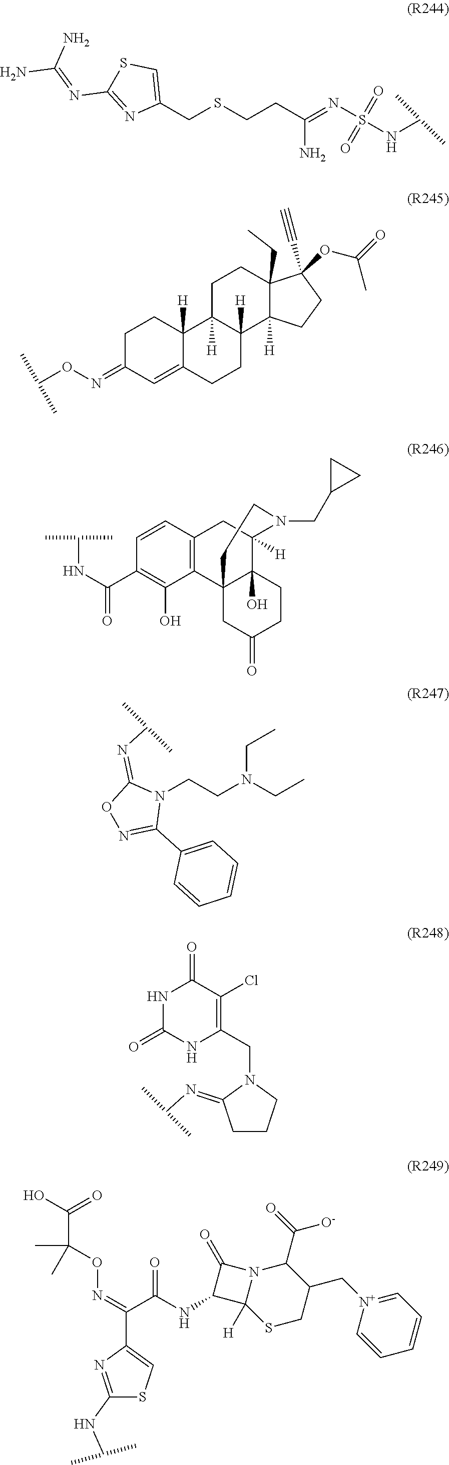

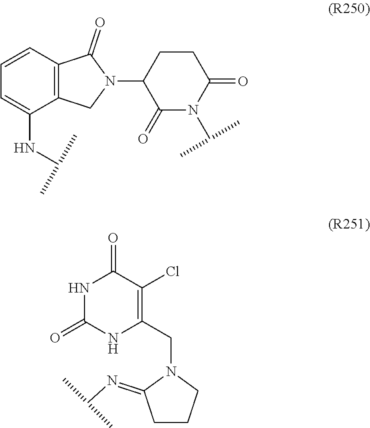

In another embodiment, the prodrug has a formula R1₂-R2, wherein: each R1- is a prodrug moiety selected from any one of R10-R105; and —R2 is R252 or R253, wherein the dashed line ( [symbol] ) is a bond.

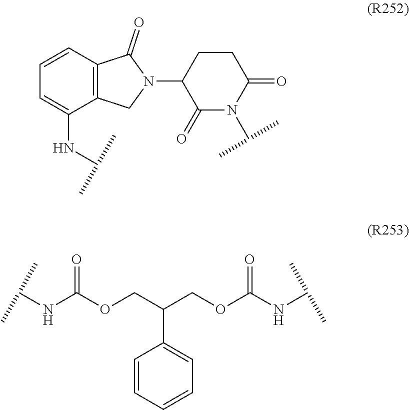

In one embodiment, the prodrug is compound of R252 or R253.

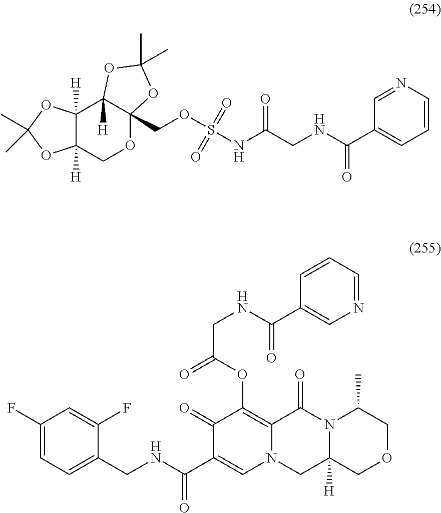

In one embodiment, the composition is a pharmaceutical composition. In a further embodiment, the pharmaceutical composition comprises a pharmaceutically acceptable excipient.

The prodrugs of the present invention comprise an API with a modification to at least one functional group, e.g., a hydroxy, present on the free form (free acid or free base) of the API, whereby the functional group is substituted with a prodrug moiety of the present invention.

In one embodiment, the composition is a salt or co-crystal of the prodrug.

The compositions of the present invention further include prodrugs that have at least one improvement selected from: increased stability (e.g., in water, gastric or intestinal fluid, or at elevated temperature, e.g. at 100° C.), solubility, dissolution, oral bioavailability, Cmax, absorption, permeability into the enterocyte, transfer from the enterocyte to the blood (portal vein), transport into liver, uptake by hepatic cells; or reduced time to Tmax, low intra-subject variability (including reduced food effect on absorption), lymphatic circulation or conversion to the parent xanthophyll in gastric and intestinal fluid (either chemically or enzymatically); or enhanced safety in human and animal disease treatment, including low liver toxic potential, preferential clearance mechanism sparing liver metabolism in advanced liver disease with loss of hepatic function.

In one embodiment, the composition is a pharmaceutical composition comprising a pharmaceutically acceptable excipient.

In other embodiments, a pharmaceutical composition of the present invention is delivered to a subject via an oral, parenteral, enteral, or a topical route of administration, preferably oral administration. In one embodiment, the pharmaceutical composition is an oral dosage form. In various embodiments, the oral dosage form is a solid, liquid, or semi-solid oral dosage form.

A pharmaceutical formulation of the present invention may be in any pharmaceutical dosage form. The pharmaceutical formulation may be, for example, a tablet, capsule, extrudate, nanoparticulate material, e.g., granulated particulate material or a powder, a lyophilized material for reconstitution, liquid suspension, injectable suspension or solution, suppository, or topical or transdermal preparation or patch.

In another embodiment, the invention provides for a unit dose of the pharmaceutical composition of the present invention. In the above methods of treating, the treatment is carried out by one or more unit doses administered per day. The daily dose of the pharmaceutical composition is preferably approximately 0.1-50 mg/kg body weight, 0.1-10 mg/kg body weight, 0.1-5 mg/kg body weight, 0.1-2 mg/kg body weight, 5-10 mg/kg body weight, 10-20 mg/kg body weight, 20-30 mg/kg body weight, 30-40 mg/kg body weight, 40-50 mg/kg body weight, 50-60 mg/kg body weight or 10-1000 mg, in particular 10-200 mg, 10-100 mg, 5-50 mg, 50-100 mg, 100-150 mg, 150-200 mg, 200-300 mg, 300-400 mg, 400-500 mg, 500-750 mg, 750-1000 mg, 1000-1500 mg, 1500-2000 mg, 2000-2500 mg, 2500-3000 mg, 3000-3500 mg, or 3500-4000 mg, in each case calculated as the parent API. The doses can be administered in any convenient dosing schedule to achieve the stated beneficial effects. For example, the doses can be taken 1, 2 or 3 times daily.

In one embodiment, the unit dose comprises 1-100 μg, 100-500 μg, 500 μg-1 mg, 1-5 mg, 5-15 mg, 10-40 mg (e.g., 15-40 mg, 20-30 mg, 20-40 mg, 25-40 mg, 30-40 mg, 35-40 mg, 10-35 mg, 10-30 mg, 10-25 mg, 10-20 mg, 10-15 mg), 30-60 mg (e.g., 35-60 mg, 40-60 mg, 45-60 mg, 50-60 mg, 55-60 mg, 30-55 mg, 30-50 mg, 30-45 mg, 30-40 mg, 30-35 mg), or 10-100 mg, 200-300 mg, 300-400 mg, 400-500 mg, 500-750 mg, 750-1000 mg, or 1000-1500 mg.

### Methods of Treating

In addition to promoting general state of health, liver health, cardiovascular health or improving inflammatory health the compositions of the present invention may be used to treat a variety of diseases and conditions. Accordingly, another aspect provides for a method of treating a subject having a disease or disorder that would benefit from the administration of a prodrug of the present invention, said method comprising the step of administering to said subject an effective amount of said prodrug. The prodrugs of the present invention are therapeutically useful for the treatment and/or prevention of a disease for which the parent drug is indicated, e.g., fibrotic diseases such as NASH. Accordingly, in another aspect, the invention also relates to: methods of treating a subject using a prodrug of the present invention or salt or co-crystal thereof; methods of treating a subject using a pharmaceutical composition comprising the prodrug or salt or co-crystal thereof and a pharmaceutically acceptable excipient; and use of the prodrug or prodrug pharmaceutical composition as a medicament for the treatment of a disease or condition as described herein.

### EXAMPLES

### Compound Synthesis

The prodrugs of the present invention can be manufactured in principle according to synthetic methods known per se for esterification, amidation, thioesterification, N-alkylation, O-alkylation or S-alkylation according to the nature of the group R1. For example, the API is reacted with the pertinent acid RCOOH as such or as its acid chloride RCOCl, acid anhydride (RCO)2O or chloromethylester RCOOCH₂Cl. These processes for producing the prodrugs represent a further aspect of the present invention.

In the case of esterification, amidation, or thioesterification with an acid, acid chloride or acid anhydride, or N-alkylation, O-alkylation or S-alkylation with chloromethylester, the reaction is generally conducted in an inert solvent and in the presence of an organic base. The solvent to be used is not particularly limited, as long as it is inert to the present reaction, but it may be, for example, an aliphatic hydrocarbon such as hexane, heptane, ligroin and petroleum ether; an aromatic hydrocarbon such as benzene, toluene and xylene; a halogenated hydrocarbon such as methylene chloride, chloroform, 1,2-dichloroethane and carbon tetrachloride; an ether such as diethyl ether, di-isopropyl ether, tetrahydrofuran, dioxane, dimethoxy-ethane and diethylene glycol dimethyl ether; a ketone such as acetone; an amide such as formamide, dimethylformamide, dimethylacetoamide and hexamethylphosphoric acid triamide; a sulfoxide such as dimethyl sulfoxide; or sulfolane, and it is preferably a halogenated hydrocarbon, an ether or an amide and most preferably methylene chloride, chloroform, tetrahydrofuran, dioxane or dimethylformamide. The solvents can be used alone or as a combination.

The base to be used is, for example, an alkali metal carbonic acid salt such as lithium carbonate, sodium carbonate and potassium carbonate; an alkali metal hydrogen carbonic acid salt such as lithium hydrogen carbonate, sodium hydrogen carbonate and potassium hydrogen carbonate; an alkali metal hydride such as lithium hydride, sodium hydride and potassium hydride; an alkali metal hydroxide such as lithium hydroxide, sodium hydroxide and potassium hydroxide; an alkali metal alkoxide such as lithium methoxide, sodium methoxide, sodium ethoxide and potassium t-butoxide; or an organic amine such as triethyl-amine, tributylamine, N,N-diisopropylethylamine, N-methylmorpholine, pyridine, 4-(N,N-dimethylamino)pyridine, N,N-dimethylaniline, N,N-diethylaniline, 1,5-diazabicyclo [4.3.0]non-5-ene, l,4-diazabicyclo[2.2.2]octane (DABCO) and l,8-diazabicyclo[5,4.0]-7-undecene (DBU) and it is preferably an organic amine and most preferably triethylamine, 4-(N,N-dimethylamino)pyridine or N,N-diisopropylethylamine. The bases can be used alone or as a combination.

The molar ratio of API:acid chloride or acid anhydride:base is conveniently in the range of 1:2-6:2-10. It has been found to be advantageous to conduct the esterification, amidation, and thioesterification under an inert atmosphere, preferably using nitrogen or argon as the inert gas.

Where the acid itself is used to for esterification or amidation of the API, the conditions are generally similar to those employed for esterifications and amidations with an acid chloride or anhydride in respect of the solvent/dispersion medium and reaction temperatures. However, an active agent is generally employed in combination or not with a base. The active agent to be used may be, for example, an N-hydroxy compound such as N-hydroxysuccinimide, 1-hydroxybenzotriazole and N-hydroxy-5-norbornen-2,3-dicarboxyimide; a disulfide compound such as dipyridyl disulfide; a carbodiimide such as N,N′-diisopropylcarbodiimide, dicyclohexylcarbodiimide, 1-ethyl-3-(3-di-methylaminopropyl)carbodiimide hydrochloride and bis-(trimethylsilyl)carbodiimide; 1,1′-carbonylbis-1H-imidazole; 4-(4,6-dimethoxy-1,3,5-triazin-2-yl)-4-methylmorpholinium chloride (DMTMM), diphenylphosphoric acid azide, hexafluorophosphoric acid benzotriazol-1-yloxy-tris(dimethylamino)phosphonium or triphenylphosphine and it is preferably N,N′-diisopropylcarbodiimide, dicyclohexylcarbodiimide, 1-ethyl-3-(3-dimethylaminopropyl)carbodiimide hydrochloride, 4-(4,6-dimethoxy-1,3,5-triazin-2-yl)-4-methylmorpholinium chloride (DMTMM), diphenyl phosphoric acid azide or 1,1′-carbo-nylbis-1H-imidazole and most preferably N,N′-diisopropylcarbodiimide, 1-ethyl-3-(3-dimethylaminopropyl)-carbodiimide hydrochloride, 4-(4,6-dimethoxy-1,3,5-triazin-2-yl)-4-methylmorpholinium chloride (DMTMM) or 1,1′-carbonylbis-1H-imidazole.]

The molar ratio of API:carboxylic acid-active agent is conveniently in the range of 1:2-6:2-7. The pertinent acids RCOOH, acid chlorides RCOCl, acid anhydrides, (RCO)₂O and chloromethylester RCOOCH2Cl, used as starting materials in the above-described processes for producing the prodrugs are either known compounds, or can be readily produced by processes analogous to the processes for producing the related known starting materials.

In all these cases the product, i.e., prodrugs, can be isolated and purified by methods known per se, e.g., by adding a solvent to induce the separation of the crude product from the mixture after reaction, and crystallization of the collected crude product. Column chromatography may further be employed in purification.

### Example 1

### Prodrug of Voriconazole (Ester of Nicotinuric Acid)

1.3 mmol of voriconazole, 462 mg (2.56 mmol) nicotinuric acid and 20 mL of dichloromethane were added to a 50 mL round bottom flask under nitrogen atmosphere. The mixture was stirred and cooled in an ice bath. 1217 mg (6.35 mmol) of 1-Ethyl-3-(3-dimethylaminopropyl) carbodiimide and 844 mg (6.91 mmol) of 4-Dimethylaminopyridine was added. The reaction was allowed to reach room temperature and the mixture was stirred for 24 hours until the free API was consumed. The reaction was monitored by silica gel TLC. The reaction solution was extracted with a citric acid solution and water. The dichloromethane layer was dried over anhydrous magnesium sulfate. The solvent then was removed under reduced pressure via rotary evaporation. The residue was purified by preparative HPLC. (257) was obtained, 60% yield.

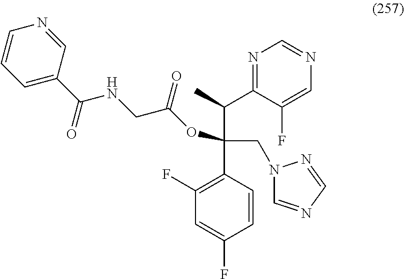 [SMILES: C[C@@H](c1ncncc1F)[C@@](Cn1cncn1)(OC(=O)CNC(=O)c1cccnc1)c1ccc(F)cc1F]

### Example 2

### Prodrug of Voriconazole (Ester of 3-(pyridin-3-ylformamido)propanoic acid)

The precursor 3-(pyridin-3-ylformamido)propanoic acid used for the esterification of voriconazole was prepared by reacting pyridine-3-carbonyl azide with the aminoacid (β-alanine) according to the following procedure: A 100 mL one-necked round bottom flask was charged with 1.707 g (13.87 mmol) of nicotinic acid and 45 mL of dry dichloromethane under nitrogen atmosphere. The solution was stirred and cooled in an ice bath while 3.357 g (17.51 mmol) of 1-ethyl-3-(3-dimethylaminopropyl) carbodiimide was added. After a further 15 min in the ice bath, 2.627 g (40.41 mmol) of sodium azide was added. The ice bath was removed, and the reaction mixture was stirred for 20 h at room temperature. The reaction was monitored by silica gel TLC. The reaction solution was washed with brine and the bottom organic layer was removed. The remaining aqueous layer was extracted with dichloromethane. The dichloromethane fractions were combined and washed with water, the bottom organic layer was dried over anhydrous magnesium sulfate and filtered. The solvent was removed under vacuum by rotary evaporation at 30° C. 2546 mg of crude was obtained and the content of pyridine-3-carbonyl azide in the crude was ˜80% w/w (¹H NMR (CDCl₃, 200 MHz): δ 7.44 (dd, 1H, J=4.7, 7.9 Hz), 8.30 (ddd, 1H, J=1.9, 2.0, 8.0 Hz), 8.84 (dd, 1H, J=1.6, 4.8 Hz), 9.17 (d, 1H, J=1.9 Hz). A 50 mL one-necked round bottom flask was charged with 5 mL of a solution of β-alanine (1.213 g, 13.5 mmol) in carbonate buffer 0.5 M pH 10 under nitrogen atmosphere and at room temperature. 2.0 g (13.5 mmol) of pyridine-3-carbonyl azide (2.4 g of the crude) was dissolved in 1 mL of dichloromethane, and the solution was added in small aliquots every two minutes to the aminoacid solution. The pH was measured with a test strip after each addition and adjusted with carbonate buffer 0.5M, if needed, to pH 8. The reaction was monitored by silica gel TLC. After 40 minutes, 1.9 mL of a solution 10 M of hydrochloric acid was added until reaching pH 4 and it was cooled to 4° C. (rate of cooling: 1° C. per minute). The product crystallized and the solid was isolated by filtration. The crystals were washed with a hydrochloric acid aqueous solution pH 4 (3×5 mL) and dried. 1.127 g (5.8 mmol) of 3-(pyridin-3-ylformamido)propanoic acid were obtained, 43% yield. ¹H NMR (D₂O, 200 MHz): δ 2.60 (t, 2H), 3.58 (t, 2H), 7.61 (m, 1H), 8.22 (m, 1H), 8.64 (m, 1H), 8.80 (s, 1H).

1.3 mmol of voriconazole, 2.56 mmol of 3-(pyridin-3-ylformamido)propanoic acid and 20 mL of dichloromethane were added to a 50 mL round bottom flask under nitrogen atmosphere. The mixture was stirred and cooled in an ice bath. 1217 mg (6.35 mmol) of 1-Ethyl-3-(3-dimethylaminopropyl) carbodiimide and 844 mg (6.91 mmol) of 4-Dimethylaminopyridine was added. The reaction was allowed to reach room temperature and the mixture was stirred for 24 hours until the free API was consumed. The reaction was monitored by silica gel TLC. The reaction solution was extracted with citric acid solution and water. The dichloromethane layer was dried over anhydrous magnesium sulfate. The solvent then was removed under reduced pressure via rotary evaporation. The residue was purified by preparative HPLC. (258) was obtained, 50% yield.

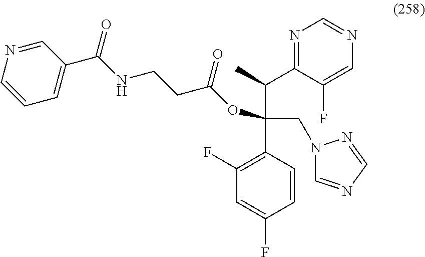 [SMILES: C[C@@H](c1ncncc1F)[C@@](Cn1cncn1)(OC(=O)CCNC(=O)c1cccnc1)c1ccc(F)cc1F]

### Example 3

### Prodrug of Aripiprazole (N-Alkylation with Chloromethyl Ester of Nicotinuric Acid)

A mixture of aripiprazole (0.45 grams, 0.01 mol) in 1,4-dioxane (80 mL) was sonicated to dissolve the aripiprazole completely, and then treated with NaH (3.8 g, 0.095 mol, 60% dispersion) in one portion. After stirring this reaction mixture for 15 minutes at room temperature, the reaction mixture was treated dropwise with dry chloromethyl ester of nicotinuric acid (0.03 mol) and a catalytic amount of sodium iodide(0.005 mol). The resultant cloudy mixture was heated to 90° C. for 2 hours, cooled to ambient temperature and poured into water. The product was extracted with dichloromethane, and the combined organic layers washed with brine, dried over magnesium sulfate, filtered and concentrated under reduced pressure. Column chromatography over silica gel provided the desired product (259) (70% yield).

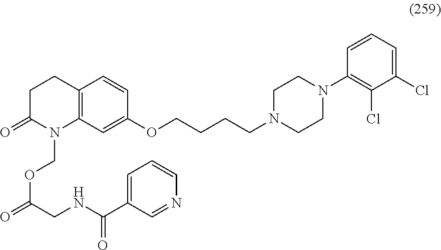 [SMILES: O=C(CNC(=O)c1cccnc1)OCN1C(=O)CCc2ccc(OCCCCN3CCN(c4cccc(Cl)c4Cl)CC3)cc21]

1. A composition comprising a prodrug, wherein: said prodrug comprises an active pharmaceutical ingredient substituted with a prodrug moiety for a hydrogen atom; wherein said prodrug moiety is selected from any one of R10-R105.

2-69. (canceled)
